(19) United States
(12) Patent Application Publication
(10) Pub. No.: US 2024/0354569 A1
(43) Pub. Date: Oct. 24, 2024

(54) METHOD, PROGRAM AND DEVICE FOR IMPROVING QUALITY OF MEDICAL DATA BASED ON DEEP LEARNING

(71) Applicant: AIRS MEDICAL INC., Seoul (KR)

(72) Inventors: Hyeonsoo KIM, Seoul (KR); Jonyoung YANG, Seoul (KR)

(21) Appl. No.: 18/721,812

(22) PCT Filed: Jun. 20, 2023
(86) PCT No.: PCT/KR2023/008542

§ 371 Date: Jun. 19, 2024

(30) Foreign Application Priority Data
Jul. 6, 2022 (KR) 10-2022-0083077

(51) Int. Cl. G06N 3/08

(52) U.S. Cl. CPC G06N 3/08

(57) ABSTRACT

According to an embodiment of the present, there are disclosed a method, program and device for improving the quality of medical data based on deep learning that is performed by a computing device. The method includes: acquiring at least one of the noise ratio of label data having a correlation with the noise of input data for the training of a neural network model, the resolution of the input data, and the resolution of the label data; and training the neural network model based on at least one of the input data, the label data, the noise ratio, the resolution of the input data, and the resolution of the label data.

### TECHNICAL FIELD

The present disclosure relates to deep learning technology, and more particularly to training and inference methods for a deep learning model that allows the degree of improvement in the quality of medical data to be adjusted.

### BACKGROUND ART

When an artificial neural network for improving the quality of data is trained, the degree of improvement in the quality of data may vary depending on how input data and label data are configured. In general, an artificial neural network generates output data, determined through training, for input data. For example, an artificial neural network that has been trained to output reduced noise and a doubly-improved signal-to-noise ratio may output an image having reduced noise and a doubly-improved signal-to-noise ratio for a specific input image. In this case, when a user desires to obtain an output image having reduced noise and a triply-improved signal-to-noise ratio, there is required a separate artificial neural network trained to output a triply-improved signal-to-noise ratio. When multiple artificial neural networks are constructed according to the degree of alleviation of noise as described above, a lot of storage space and computing power are inevitably required in a system. Therefore, in order to effectively improve the quality of data based on deep learning, there is a need for an idea that allows a single neural network to adjust the quality of output data even without the construction of multiple neural networks.

### DISCLOSURE

### Technical Problem

The present disclosure has been conceived in response to the above-described background technology, and an object of the present invention is to provide training and inference methods for a deep learning model that allows an image having the quality improved in accordance with the ratio desired by a user to be generated.

However, the objects to be accomplished by the present disclosure are not limited to the objects mentioned above, and other objects not mentioned may be clearly understood based on the following description.

### Technical Solution

According to an embodiment of the present disclosure for achieving the above-described object, there is disclosed a method of improving the quality of medical data based on deep learning that is performed by a computing device. The method includes: acquiring at least one of the noise ratio of label data having a correlation with the noise of input data for the training of a neural network model, the resolution of the input data, and the resolution of the label data; and training the neural network model based on at least one of the input data, the label data, the noise ratio, the resolution of the input data, and the resolution of the label data.

Alternatively, the signal intensity of the label data may correspond to the signal intensity of the input data, and the noise of the label data may be different from the noise of the input data.

Alternatively, the noise of the label data may be determined through a linear combination of dependent noise having a correlation with the noise of the input data and independent noise having no correlation with the noise of the input data.

Alternatively, training the neural network model based on at least one of the input data, the label data, the noise ratio, the resolution of the input data, and the resolution of the label data may include training the neural network model so that the neural network model improves at least one of the noise and resolution of the input data by using at least one of the noise ratio, the resolution of the input data, and the resolution of the label data.

Alternatively, the neural network model may include: a first neural network for extracting features from the input data and generating output data of the neural network model; and a second neural network for adjusting at least one of the noise and resolution of the output of the first neural network based on at least one of the noise ratio, the resolution of the input data, and the resolution of the label data.

Alternatively, the second neural network may perform an operation of applying at least one of the noise ratio, the resolution of the input data, and the resolution of the label data to the output of the first neural network in accordance with the number of channels of the first neural network.

Alternatively, the neural network model may include a third neural network for extracting features from the input data and generating output data of the neural network model in which at least one of the noise and resolution thereof has been adjusted.

Alternatively, the third neural network may perform an operation of generating output data including a first channel representing a signal without noise and a second channel representing noise adjusted according to the noise ratio with no signal present.

Meanwhile, according to an embodiment of the present disclosure for achieving the above-described object, there is disclosed a method of improving the quality of medical data based on deep learning that is performed by a computing device. The method includes: acquiring medical data and an adjustment ratio for at least one of the noise and resolution of the medical data; and alleviating or improving at least one of the noise and resolution of the medical data based on the adjustment ratio by using a pre-trained neural network model. In this case, the neural network model is pre-trained based on at least one of input data for the training of the neural network model, label data, the noise ratio of the label data having a correlation with the noise of the input data, the noise ratio, the resolution of the input data, and the resolution of the label data.

According to an embodiment of the present disclosure for achieving the above-described object, there is disclosed a computer program stored in a computer-readable storage medium. The computer program performs operations for improving the quality of medical data based on deep learning when executed on one or more processors. In this case, the operations include operations of: acquiring at least one of the noise ratio of label data having a correlation with the noise of input data for the training of a neural network model, the resolution of the input data, and the resolution of the label data; and training the neural network model based on at least one of the input data, the label data, the noise ratio, the resolution of the input data, and the resolution of the label data.

According to an embodiment of the present disclosure for achieving the above-described object, there is disclosed a computing device. The computing device includes: a processor including at least one core; memory including program code executable on the processor; and a network unit configured to acquire at least one of the noise ratio of label data having a correlation with the noise of input data for the training of a neural network model, the resolution of the input data, and the resolution of the label data. In this case, the processor trains the neural network model based on at least one of the input data, the label data, the noise ratio, the resolution of the input data, and the resolution of the label data.

### Advantageous Effects

The present disclosure may provide training and inference methods for a deep learning model that allows an image having the quality improved in accordance with the ratio desired by a user to be generated.

### DESCRIPTION OF DRAWINGS

FIG. 1 is a block diagram of a computing device according to an embodiment of the present disclosure;

FIG. 2 is a block diagram showing a process of improving the quality of medical data based on deep learning according to an embodiment of the present disclosure;

FIG. 3 is a conceptual diagram showing the structure of a neural network model according to an embodiment of the present disclosure;

FIG. 4 is a conceptual diagram showing the structure of a neural network model according to an alternative embodiment of the present disclosure;

FIG. 5 is a flowchart showing a method of training a neural network model according to an embodiment of the present disclosure; and

FIG. 6 is a flowchart showing a method of improving the quality of medical data using a neural network model according to an embodiment of the present disclosure.

### MODE FOR INVENTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “N-th (N is a natural number)” used herein can be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

Meanwhile, the term “module” or “unit” used herein may be understood as a term r to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of components of a computing device, an application program performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

“Data” used herein may include an “image.” The term “image” used herein may refer to multidimensional data composed of discrete image elements. In other words, “image” may be understood as a term referring to a digital representation of an object that can be seen by the human eye. For example, “image” may refer to multidimensional data composed of elements corresponding to pixels in a two-dimensional image. “Image” may refer to multidimensional data composed of elements corresponding to voxels in a three-dimensional image.

The term “medical image archiving and communication system (PACS)” used herein refers to a system that stores, processes, and transmits medical images in accordance with the Digital Imaging and Communications in Medicine (DICOM) standard. For example, the “PACS” operates in conjunction with digital medical imaging equipment, and stores medical images such as magnetic resonance imaging (MRI) images and computed tomography (CT) images in accordance with the DICOM standard. The “PACS” may transmit medical images to terminals inside and outside a hospital over a communication network. In this case, meta information such as reading results and medical records may be added to the medical images.

The term “k-space” used herein may be understood as an array of numbers representing the spatial frequencies of a magnetic resonance image. In other words, the “k-space” may be understood as a frequency space corresponding to a space corresponding to coordinates of a magnetic resonance space.

The term “Fourier transform” used herein may be understood as an operation medium that enables the description of the correlation between the time domain and the frequency domain. In other words, “Fourier transform” used herein may be understood as a broad concept representing an operation process for the mutual transformation between the time domain and the frequency domain. Accordingly, the “Fourier transform” used herein may be understood as a concept that encompasses both the Fourier transform in a narrow sense, which decomposes a signal in the time domain into the frequency domain, and inverse Fourier transform, which transforms a signal in the frequency domain into the time domain.

The foregoing descriptions of the terms are intended to help to understand the present disclosure. Accordingly, it should be noted that unless the above-described terms are explicitly described as limiting the content of the present disclosure, the terms in the content of the present disclosure are not used in the sense of limiting the technical spirit of the present disclosure.

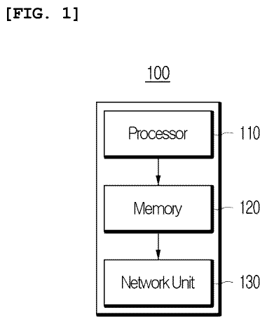

FIG. 1 is a block diagram of a computing device according to an embodiment of the present disclosure.

A computing device 100 according to an embodiment of the present disclosure may be a hardware device or part of a hardware device that performs the comprehensive processing and calculation of data, or may be a software-based computing environment that is connected to a communication network. For example, the computing device 100 may be a server that performs an intensive data processing function and shares resources, or may be a client that shares resources through interaction with a server. Furthermore, the computing device 100 may be a cloud system in which a plurality of servers and clients interact with each other and comprehensively process data. Since the above descriptions are only examples related to the type of computing device 100, the type of computing device 100 may be configured in various manners within range understandable to those skilled in the art based on the content of the present disclosure.

Referring to FIG. 1, the computing device 100 according to an embodiment of the present disclosure may include a processor 110, memory 120, and a network unit 130. However, FIG. 1 shows only an example, and the computing device 100 may include other components for implementing a computing environment. Furthermore, only some of the components disclosed above may be included in the computing device 100.

The processor 110 according to an embodiment of the present disclosure may be understood as a configuration unit including hardware and/or software for performing computing operation. For example, the processor 110 may read a computer program and perform data processing for machine learning. The processor 110 may process computational processes such as the processing of input data for machine learning, the extraction of features for machine learning, and the calculation of errors based on backpropagation. The processor 110 for performing such data processing may include a central processing unit (CPU), a general purpose graphics processing unit (GPGPU), a tensor processing unit (TPU), an application specific integrated circuit (ASIC), or a field programmable gate array (FPGA). Since the types of processor 110 described above are only examples, the type of processor 110 may be configured in various manners within a range understandable to those skilled in the art based on the content of the present disclosure.

The processor 110 may train a neural network model for improving the quality of medical data. The processor 110 may train the neural network model based on medical data and information indicating the degree of improvement in the quality of the medical data. In this case, the medical data may include a magnetic resonance signal or image acquired through accelerated imaging or accelerated simulation. Furthermore, information indicating the degree of improvement in the quality of the medical data may include the noise rate or resolution rate of the medical data. For example, the processor 110 may train the neural network model for improving at least one of the noise or resolution of the medical data based on at least one of input data for training, label data, the noise ratio of the label data, the resolution of the input data, and the resolution of the label data. In this case, the noise ratio or resolution may not be a fixed value but a value that changes depending on a user's intention. That is, the processor 110 may train the neural network model by using at least one of the noise ratio of label data, the resolution of input data, and the resolution of the label data so that the neural network model can output medical data having the quality improved in accordance with the user's intention.

Meanwhile, in the present disclosure, the accelerated imaging may be understood as an imaging technique that shortens imaging time by reducing the number of excitations (NEX) for a magnetic resonance signal compared to general imaging. The number of excitations may be understood as the number of repetitions when lines of a magnetic resonance signal are repeatedly acquired in the k-space domain. Accordingly, as the number of excitations increases, the imaging time required to image a magnetic resonance image may increase proportionally. That is, in the case where the number of excitations decreases when a magnetic resonance image is imaged, the accelerated imaging for which the imaging time required to image a magnetic resonance image is shortened may be implemented.

In the present disclosure, the accelerated imaging may be understood as an imaging technique that shortens imaging time by increasing the acceleration factor compared to regular imaging. The acceleration factor is a term used in a parallel imaging technique, and may be understood as a value obtained by dividing the number of signal lines fully sampled in the K-space domain by the number of signal lines sampled through imaging. For example, an acceleration factor of 2 can be understood as obtaining a number of signal lines equivalent to half of the number of fully sampled signal lines when lines are obtained by sampling a magnetic resonance signal in the phase encoding direction. Accordingly, as the acceleration factor increases, the imaging time of a magnetic resonance image may decrease proportionally. That is, when the acceleration factor is increased during the imaging of a magnetic resonance image, accelerated imaging in which magnetic resonance image imaging time is shortened may be implemented.

In the present disclosure, the accelerated imaging may be understood as an imaging technique that shortens imaging time by increasing the acceleration factor compared to regular imaging. The acceleration factor is a term used in parallel imaging techniques, and may be understood as a value obtained by dividing the number of signal lines fully sampled in k-space by the number of signal lines sampled through imaging. For example, the fact that the acceleration factor is 2 may be understood as obtaining a number of signal lines equal to half of the number of fully sampled signal lines when obtaining lines by sampling a magnetic resonance signal in the phase encoding direction. Accordingly, as the acceleration factor increases, the imaging time required to image a magnetic resonance image may decrease proportionally. That is, in the case where the acceleration factor increases when a magnetic resonance image is imaged, the accelerated imaging in which the imaging time required to image a magnetic resonance image is shortened may be implemented.

In the present disclosure, the accelerated imaging may be understood as an imaging technique that generates a magnetic resonance image by acquiring a sub-sampled magnetic resonance signal. In this case, the subsampling may be understood as an operation of sampling a magnetic resonance signal at a sampling rate lower than the Nyquist sampling rate. Accordingly, the medical data of the present disclosure may be an image acquired by sampling a magnetic resonance signal at a sampling rate lower than the Nyquist sampling rate.

The accelerated simulation of the present disclosure may be understood as an operation technique for under-sampling three-dimensional k-space data generated through regular or accelerated imaging. In this case, the under-sampling may be understood as a method of processing a magnetic resonance signal at a lower sampling rate based on the three-dimensional k-space data to be processed. For example, the accelerated simulation may include an operation technique that generates subsampled three-dimensional k-space data based on fully sampled three-dimensional k-space data. Furthermore, the accelerated simulation may include an operation technique that samples a magnetic resonance signal at a lower sampling rate based on subsampled three-dimensional k-space data. In addition to these examples, the accelerated imaging technique described above may be applied to the accelerated simulation without change. The acceleration simulation may be performed by the processor 110 of the present disclosure, or may be performed through a separate external system.

However, since the above description of accelerated imaging or accelerated simulation is only an example, the concept of accelerated imaging may be defined in various manners within a range understandable to those skilled in the art based on the content of the present disclosure. The processor 110 may improve low-quality medical data to high quality by using the neural network model pre-trained as described above. The processor 110 may improve the quality of medical data based on the quality improvement ratio of the medical data by using the pre-trained neural network model. The processor 110 may generate medical data having the quality improved in accordance with the quality improvement ratio by inputting medical data and the quality improvement ratio of the medical data to the neural network model. For example, the processor 110 may generate medical data in which at least one of the noise and resolution of the medical data has been improved according to an adjustment ratio based on the adjustment ratio for at least one of the noise and resolution of the medical data by using the neural network model. In this case, the neural network model may be pre-trained based on at least one of input data and label data for the training of the neural network model, the noise ratio of the label data, the resolution of the input data, and the resolution of the label data. That is, the processor 110 may generate medical data in which at least one of the noise and resolution has been improved at the ratio desired by a user by using the neural network model pre-trained as described above.

The memory 120 according to an embodiment of the present disclosure may be understood as a configuration unit including hardware and/or software for storing and managing data that is processed in the computing device 100. That is, the memory 120 may store any type of data generated or determined by the processor 110 and any type of data received by the network unit 130. For example, the memory 120 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 120 may include a database system that controls and manages data in a predetermined system. Since the types of memory 120 described above are only examples, the type of memory 120 may be configured in various manners within a range understandable to those skilled in the art based on the content of the present disclosure.

The memory 120 may structure, organize, and manage data necessary for the processor 110 to perform computation, the combination of data, and program codes executable on the processor 110. For example, the memory 120 may store medical data received through the network unit 130, which will be described later. The memory 120 may store program code configured to perform operation so that a neural network model processes medical data, program code configured to operate the processor 110 to generate image data based on the feature interpretation (or feature inference) of the neural network model, and k-space data, image data, etc. generated as the program code is executed.

The network unit 130 according to an embodiment of the present disclosure may be understood as a configuration unit that transmits and receives data through any type of known wired/wireless communication system. For example, the network unit 130 may perform data transmission and reception using a wired/wireless communication system such as a local area network (LAN), a wideband code division multiple access (WCDMA) network, a long term evolution (LTE) network, the wireless broadband Internet (WiBro), a 5th generation mobile communication (5G), a ultra wide-band wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 130 may be applied in various manners other than the above-described examples.

The network unit 130 may receive data necessary for the processor 110 to perform computation through wired/wireless communication with any system or client or the like. Furthermore, the network unit 130 may transmit data generated through the computation of the processor 110 through wired/wireless communication with any system or client or the like. For example, the network unit 130 may receive medical data through communication with a PACS, a cloud server that performs tasks such as the standardization of medical data, or a computing device. The network unit 130 may transmit image data corresponding to the output of the neural network model, verification data determined during the operation process of the processor 110, etc. through communication with the above-described PACS, cloud server, or computing device.

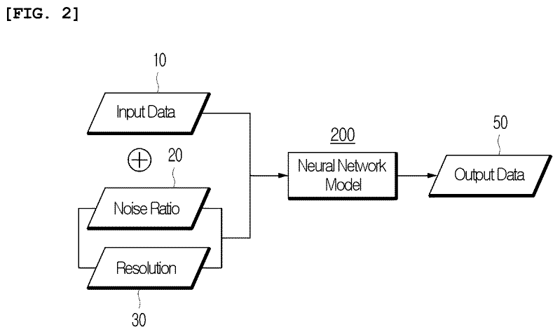

FIG. 2 is a block diagram showing a process of improving the quality of medical data based on deep learning according to an embodiment of the present disclosure.

Referring to FIG. 2, the processor 110 of the computing device 100 according to an embodiment of the present disclosure may train a neural network model 200 to alleviate or improve at least one of the noise and resolution of input data 10 based on at least one of the input data 10, a noise ratio 20, and a resolution 30. The processor 110 may generate output data 50 in which at least one of the noise and resolution thereof has been alleviated or improved from the input data 10 by inputting at least one of the input data 10, the noise ratio 20, and the resolution 30 to the neural network model 200. The processor 110 may train the neural network model 200 by comparing errors between the output data 50 and the label data and adjusting a parameter of the neural network model 200 according to the results of the comparison. In this case, the noise ratio 20 may be the noise ratio of label data that has a correlation with the noise of the input data 10 for the training of the neural network model 200. Furthermore, the resolution 30 may be at least one of the resolution of the input data 10 and the resolution of the label data. The processor 110 may effectively train the neural network model 200 even in the absence of training data having various noise or resolution distributions by utilizing at least one of the noise ratio 20 and the resolution 30 for training. Moreover, the processor 110 may efficiently generate the output data 50 having the quality improved in accordance with the ratio desired by a user by using the trained neural network model 200.

The input data 10 and label data used for the training of the neural network model 200 of the present disclosure may correspond to each other in terms of intensity, but may be independent of each other in terms of noise. In other words, the signal intensity of the input data 10 for the training of the neural network model 200 may correspond to the signal intensity of the label data. In contrast, at least one of the intensity and distribution of noise of the label data may be different from at least one of the intensity and distribution of noise of the input data 10. In this case, the noise of the label data may be determined through a linear combination of the dependent noise that has a correlation with the noise of the input data 10 and the independent noise that has no correlation with the noise of the input data 10.

For example, when the input data 10 is represented by S+n1, the label data may be represented by S+A*n1+B*n2. In this case, S denotes the signal, n1 denotes the noise of the input data 10, A*n1 denotes dependent noise having a correlation with the noise of the input data 10, and B*n2 denotes independent noise having no correlation with the noise of the input data 10. Furthermore, A and B are coefficients for the linear combination of noise, and the sum of the two coefficients may be 1 (A+B=1). In other words, the label data may have the same signal intensity as the input data 10, but may be data in which the dependent noise has a ratio of A and the independent noise has a ratio of B with respect to the total noise. In this case, A is the noise ratio of the label data that has a correlation with the noise of the input data 10 for the training of the neural network model 200, and may be used as an input for the training or inference of the neural network model 200.

When the input data 10 and the label data, which correspond to each other in terms of signal intensity but are independent of each other in terms of noise, are used for the training of the neural network model 200 in this way, the neural network model 200 including a single neural network may be more accurately trained with the aim of alleviating noise according to the ratio desired by a user.

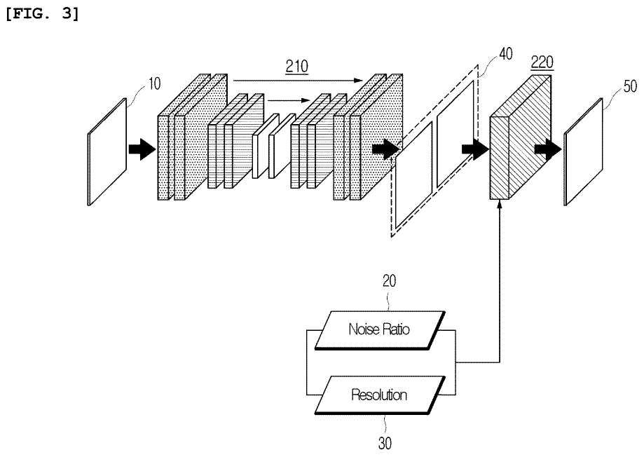

FIG. 3 is a conceptual diagram showing the structure of a neural network model according to an embodiment of the present disclosure.

Referring to FIG. 3, the neural network model 200 according to an embodiment of the present disclosure includes a first neural network 210 for extracting features from the input data 10 and generating output data 50 of the neural network model 200, and a second neural network 220 for adjusting at least one of the noise and resolution of the output 40 of the first neural network 210 based on at least one of the noise ratio 20 and the resolution 30. For example, the first neural network 210 may be U-NET, which includes a convolutional neural network optimized for extracting features of an image. The second neural network 220 may include a fully connected layer (FCL) configured to perform additional operation on the output of the first neural network 210. However, since the types of first and second neural networks 210 and 220 are only examples, the types of neural networks may be configured in various manners that can be understood by those skilled in the art based on the content of the present disclosure.

According to an embodiment of the present disclosure, the first neural network 210 may generate the data 40 corresponding to the output data 50 of the neural network model 200 by analyzing the characteristics of the input data 10. The second neural network 220 performs an operation of applying at least one of the noise ratio 20 and the resolution 30 to the output 40 of the first neural network 210 according to the number of channels of the first neural network 210. As shown in FIG. 3, when the number of channels of the first neural network 210 is 2, the second neural network 220 may generate the output data 50 in which at least one of noise and resolution has been alleviated or improved compared to the input data 10 by applying at least one of the noise ratio 20 and the resolution 30 to each channel of the first neural network 210. In this case, “apply” may be understood as a task of performing a mathematical operation according to the value indicated by the noise ratio 20 or resolution 30. For example, the second neural network 220 may generate the output data 50 having alleviated noise through an operation that multiplies the noise ratio 20 by each channel constituting the output 40 of the first neural network 210. However, since the multiplication operation is only an example, various operation methods such as a sum operation or a convolution operation may be applied within a range that can be understood by those skilled in the art based on the content of the present disclosure, in addition to the multiplication operation, which is an example described above.

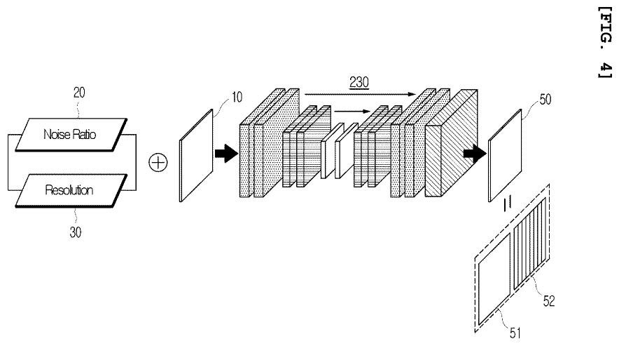

FIG. 4 is a conceptual diagram showing the structure of a neural network model according to an alternative embodiment of the present disclosure.

Referring to FIG. 4, a neural network model 200 according to an alternative embodiment of the present disclosure may include a third neural network 230 for extracting features from input data 10 and generating output data 50 in which at least one of noise and resolution has been adjusted. The third neural network 230 may perform an operation to generate output data including a first channel 51 representing a signal without noise and a second channel 52 representing the noise adjusted according to at least one of the noise ratio 20 and the resolution 30 with no signal present. That is, unlike in FIG. 3, the third neural network 230 according to an alternative embodiment of the present disclosure may be trained such that the output data 50 includes a combination of the first channel representing a signal and the second channel representing noise, and may generate the output data 50 in which at least one of the noise and the resolution has been adjusted.

For example, the third neural network 230 may generate the data 40 corresponding to the output data 50 of the neural network model 200 by analyzing characteristics of the input data 10. In this case, the third neural network 230 may configure the output data 50 as a combination of the first channel 51 and the second channel 52. The third neural network 230 may generate a signal without noise through the first channel 51 of the output data 50 based on the results of analysis of the characteristics of the input data 10. Furthermore, the third neural network 230 may generate noise adjusted in accordance with at least one of the noise ratio 20 and the resolution 30 based on at least one of the results of analysis of the characteristics of the input data 10, the noise ratio 20, and the resolution 30. Accordingly, the third neural network 230 may perform learning so that only a signal is present without noise through the first channel 51, and may perform learning so that only noise is present with no signal present through the second channel 52. Furthermore, the third neural network 230 may generate the data 40 corresponding to one image by using the noise ratio of the first channel 51 and the second channel 52. When a target noise reduction ratio is ⅓, the third neural network 230 may generate the output data 40 by combining the first channel 51 and the second channel 52 reduced at a ⅓ ratio. That is, the third neural network 230 may generate the output data 50 having the quality improved compared to the input data 10 through an operation that separates a signal channel and a noise channel and processes the individual channels.

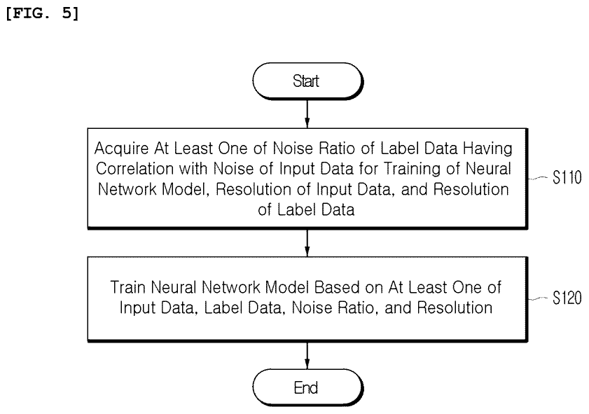

FIG. 5 is a flowchart showing a method of training a neural network model according to an embodiment of the present disclosure.

Referring to FIG. 5, the computing device 100 according to an embodiment of the present disclosure may acquire at least one of the noise ratio of label data having a correlation with the noise of input data for the training of a neural network model, the resolution of the input data, and the resolution of the label data in step S110. In this case, the term “acquisition” may be understood to mean not only receiving data over a wireless communication network connecting with an external terminal, device or system, but also generating or receiving data in an on-device form. For example, the computing device 100 may receive at least one of the noise ratio and the resolution through cloud communication with a client. The computing device 100 may be mounted on a client or server capable of user input, and may receive the input of the noise ratio or the resolution. The computing device 100 may acquire input data and label data in addition to the noise ratio and the resolution.

The computing device 110 may train the neural network model based on at least one of the input data, the label data, the noise ratio, the resolution of the input data, and the resolution of the label data in step S120. In this case, the noise ratio may be a ratio for the dependent noise of the label data that has a correlation with the noise of the input data for the training of the neural network model.

In other words, the noise ratio may be a value representing the proportion of dependent noise in the noise of the label data. The computing device 100 may train the neural network model so that the neural network model determines the degree to which the noise of the input data is alleviated according to the noise ratio and alleviates the noise. Alternatively, the computing device 100 may train the neural network model so that the neural network model determines the degree to which the resolution of the input data is improved according to the resolution and improves the resolution. Through the training of the neural network model, even when multiple neural network models are not constructed according to the degree of alleviation or improvement of noise or resolution, noise or resolution may be efficiently alleviated or improved according to the ratio desired by a user even by using a single model. Depending on which of the noise ratio and the resolution is used for training, the neural network model may be a denoising model, a super resolution model, or a model that can perform both functions.

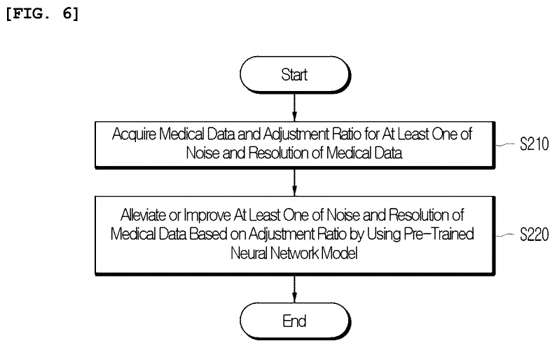

FIG. 6 is a flowchart showing a method of improving the quality of medical data using a neural network model according to an embodiment of the present disclosure.

Referring to FIG. 6, the computing device 100 according to an embodiment of the present disclosure may acquire an adjustment ratio for at least one of medical data, and the noise and resolution of the medical data in step S210. In this case, the medical data may be a magnetic resonance signal or image acquired through accelerated imaging or accelerated simulation. Furthermore, the adjustment ratio may be a numerical value for improving the noise or resolution of the medical data defined by a user. For example, the computing device 100 may receive a magnetic resonance image in the k-space domain as the medical data through cloud communication with a PACS. Furthermore, the computing device 100 may receive the adjustment ratio, input to a client by a user, through cloud communication with the client. Since the above description is only an example, the data acquisition method of the computing device 100 is not limited to the above description.

In step S220, the computing device 100 may improve at least one of the noise and resolution of the medical data based on the adjustment ratio acquired in step S210 by using the pre-trained neural network model as shown in FIG. 5. That is, the computing device 100 may generate medical data having alleviated noise or improved resolution by inputting the adjustment ratio together with the medical data to the neural network model trained through the training process of FIG. 5. By using the neural network trained according to FIG. 5 as described above, medical data having the quality improved according to the ratio desired by a user may be smoothly generated.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

1. A method of improving quality of medical data based on deep learning, the method being performed by a computing device including at least one processor, the method comprising:
acquiring at least one of a noise ratio of label data having a correlation with noise of input data for training of a neural network model, a resolution of the input data, and a resolution of the label data; and
training the neural network model based on at least one of the input data, the label data, the noise ratio, the resolution of the input data, and the resolution of the label data.

2. The method of claim 1, wherein:
a signal intensity of the label data corresponds to a signal intensity of the input data; and
noise of the label data is different from noise of the input data.

3. The method of claim 2, wherein the noise of the label data is determined through a linear combination of dependent noise having a correlation with the noise of the input data and independent noise having no correlation with the noise of the input data.

4. The method of claim 1, wherein training the neural network model based on at least one of the input data, the label data, the noise ratio, the resolution of the input data, and the resolution of the label data comprises training the neural network model so that the neural network model improves at least one of the noise and resolution of the input data by using at least one of the noise ratio, the resolution of the input data, and the resolution of the label data.

5. The method of claim 4, wherein the neural network model comprises:
a first neural network for extracting features from the input data and generating output data of the neural network model; and
a second neural network for adjusting at least one of noise and resolution of output of the first neural network based on at least one of the noise ratio, the resolution of the input data, and the resolution of the label data.

6. The method of claim 5, wherein the second neural network performs an operation of applying at least one of the noise ratio, the resolution of the input data, and the resolution of the label data to the output of the first neural network in accordance with a number of channels of the first neural network.

7. The method of claim 4, wherein the neural network model comprises a third neural network for extracting features from the input data and generating output data of the neural network model in which at least one of noise and resolution thereof has been adjusted.

8. The method of claim 7, wherein the third neural network performs an operation of generating output data including a first channel representing a signal without noise and a second channel representing noise adjusted according to the noise ratio with no signal present.

9. A method of improving quality of medical data based on deep learning, the method being performed by a computing device including at least one processor, the method comprising:
acquiring medical data and an adjustment ratio for at least one of noise and resolution of the medical data; and
alleviating or improving at least one of the noise and resolution of the medical data based on the adjustment ratio by using a pre-trained neural network model;
wherein the neural network model is pre-trained based on at least one of input data for training of the neural network model, label data, a noise ratio of the label data having a correlation with noise of the input data, the noise ratio, a resolution of the input data, and a resolution of the label data.

10. A computer program stored in a computer-readable storage medium, the computer program performing operations for improving quality of medical data based on deep learning when executed on one or more processors,
wherein the operations comprise operations of:
acquiring at least one of a noise ratio of label data having a correlation with noise of input data for training of a neural network model, a resolution of the input data, and a resolution of the label data; and
training the neural network model based on at least one of the input data, the label data, the noise ratio, the resolution of the input data, and the resolution of the label data.

11. A computing device for improving quality of medical data based on deep learning, the computing device comprising:
a processor including at least one core;
memory including program code executable on the processor; and
a network unit configured to acquire at least one of a noise ratio of label data having a correlation with noise of input data for training of a neural network model, a resolution of the input data, and a resolution of the label data;
wherein the processor trains the neural network model based on at least one of the input data, the label data, the noise ratio, the resolution of the input data, and the resolution of the label data.
